(19) United States
(12) Patent Application Publication
(10) Pub. No.: US 2014/0356386 A1
(43) Pub. Date: Dec. 4, 2014

(54) IMMUNIZATION PROTOCOL FOR DIRECTED EXPANSION AND MATURATION

(71) Applicant: Novartis AG, Basel (CH)

(72) Inventor: Philip Dormitzer, Weston, MA (US)

(73) Assignee: Novartis AG, Basel (CH)

(21) Appl. No.: 14/219,196

(22) Filed: Mar. 19, 2014

Related U.S. Application Data
(63) Continuation of application No. 13/125,526, filed on Apr. 21, 2011, now abandoned.
(60) Provisional application No. 61/196,963, filed on Oct. 21, 2008.

(51) Int. Cl. A61K 39/00

(52) U.S. Cl. CPC A61K 39/00; A61K 2039/57
USPC 424/184.1

(57) ABSTRACT

A first antigen is administered to a subject to select progenitor B cells that are suitable for subsequent production of a desirable affinity-matured antibody, and then a second antigen is administered to stimulate the expansion of B cells that produce that affinity-matured antibody. An immunization protocol is used in which two different antigens are administered (usually in series, but in some embodiment simultaneously), where the first antigen elicits an efficient germline antibody response and the second antigen elicits an efficient and desired affinity-matured antibody response.

This is a continuation application of U.S. patent application Ser. No. 13/125,526, filed Apr. 21, 2011, which is a §371 filing of PCT/IB2009/007254, filed Oct. 21, 2009, and claims the benefit under 35 U.S.C. §119(e)(1) of U.S. provisional application No. 61/196,963, filed Oct. 21, 2008, which applications are incorporated by reference herein in their entireties.

This invention relates to an immunization protocol in which two different antigens are used to immunize a subject, either sequentially or serially. One antigen stimulates expansion of a germline antibody and one or more further antigen(s) direct(s) affinity maturation.

Producing a mature (high affinity) antibody in vertebrate immune systems entails two processes: (a) an initial clonal expansion of a germ-line antibody, which has undergone VDJ recombination, followed by (b) affinity maturation of that germline antibody, involving a process of somatic hypermutation in mammals. In the initial antibody response an administered antigen stimulates the expansion of a limited set of germline antibody-expressing B cells. These germline antibodies do not necessarily have a high affinity for the administered antigen but, rather, are simply those that happen to have reasonable affinity for an epitope on the antigen. Thereafter, affinity maturation takes place, which typically involves somatic mutation of germline antibody-encoding genes that results in the production of some antibodies with higher affinity than the germline starting point. B cells that bear mutated antibodies that can bind to limiting amounts of antigen with higher affinity are more efficiently stimulated to expand.

In this overall scheme it is not necessarily true that the highest affinity affinity-matured antibody must be derived from the highest affinity germline antibody. The highest affinity antibodies may instead originate from germline antibodies that were not prominent among those displayed by the originally expanded B cells, and an antigen may inefficiently stimulate the B cells that will eventually provide the highest affinity antibodies. Furthermore, epitopes may sometimes fail to stimulate useful mature antibodies because they are unable to stimulate the correct B cells at the start of the process. An exaggerated effect of early events in the overall process may also explain the phenomenon of immunodominance.

Thus the invention uses a first antigen to select progenitor B cells that are suitable for subsequent production of a desirable affinity-matured antibody, and then uses a second antigen to stimulate the expansion of B cells that produce that affinity-matured antibody. The affinity-matured antibody has a greater affinity for a desired antigen than is exhibited by the germline antibody produced by the progenitor B cells. Thus an immunization protocol is used in which two (or, in some embodiments, more than two) different antigens are administered in series (or, in some embodiments, in combination), where the first antigen elicits an efficient germline antibody response and the second antigen elicits an efficient and desired affinity-maturation of the antibody response. The final result is an antibody that has high affinity for the desired antigen. Ideally, the invention provides an affinity-matured antibody that, if the second antigen had been administered without prior administration of the first antigen, would not have been produced in as high a titer, as rapidly, or at all.

Thus the invention provides a method for eliciting the production of an affinity-matured antibody in a subject, comprising steps of: (i) administering to the subject a first antigen that binds to a germline antibody with greater affinity than to the affinity-matured antibody; and then (ii) administering to the subject a second antigen that binds to the affinity-matured antibody with greater affinity than to the germline antibody.

The germline antibody and the affinity-matured antibody may both bind to both antigens but with different affinities. Thus the germline antibody may bind the first antigen more tightly than it binds the second antigen, whereas the affinity-matured antibody may bind the second antigen more tightly than it does to the first antigen. In other embodiments, the germline antibody may bind only to the first antigen (i.e. bind with detectable affinity) and the affinity-matured antibody may bind only to the second antigen (i.e. bind with detectable affinity). In some embodiments the germline antibody and the affinity-matured antibody may both bind a common epitope but with different affinities.

Antibody/antigen binding affinities can be determined using conventional analytical techniques e.g. using surface plasmon resonance techniques as embodied in BIAcore™ instrumentation and operated according to the manufacturer's instructions. Radioimmunoassay using radiolabeled target antigens and enzyme-linked immunosorbent assays are other methods by which binding affinity may be measured. Because the invention is concerned with eliciting a germline antibody and an affinity-matured antibody, with different affinities for desired target antigens, the precise method used to assess affinity is less important than its ability to identify differences in affinity. The germline and the affinity-matured antibodies will have a tighter binding affinity for one or both of the first and second antigens than for an arbitrary control antigen e.g. than for a host (typically human) protein.

The invention also provides a method for eliciting the production of an affinity-matured antibody in a subject, wherein the subject has previously received a first antigen that binds to a germline antibody with greater affinity than to the affinity-matured antibody, comprising a step of administering to the subject a second antigen that binds to the affinity-matured antibody with greater affinity than to the germline antibody.

The invention also provides a kit for eliciting the production of an affinity-matured antibody in a subject, comprising a first component and a second component, wherein the first component comprises an antigen that binds to a germline antibody with greater affinity than to the affinity-matured antibody, and wherein the second component comprises an antigen that binds to the affinity-matured antibody with greater affinity than to the germline antibody. The two kit components are held separately from each other in the kit and are not admixed.

The invention also provides a first antigen for use in a method for eliciting the production of an affinity-matured antibody in a subject, comprising steps of: (i) administering to the subject the first antigen, wherein the first antigen binds to a germline antibody with greater affinity than to the affinity-matured antibody; and then (ii) administering to the subject a second antigen that binds to the affinity-matured antibody with greater affinity than to the germline antibody.

The invention also provides a second antigen for use in a method for eliciting the production of an affinity-matured antibody in a subject, comprising steps of: (i) administering to the subject a first antigen that binds to a germline antibody with greater affinity than to the affinity-matured antibody; and then (ii) administering to the subject the second antigen, wherein the second antigen binds to the affinity-matured antibody with greater affinity than to the germline antibody.

The invention also provides a second antigen for use in a method for eliciting the production of an affinity-matured antibody in a subject, wherein the subject has previously received a first antigen that binds to a germline antibody with greater affinity than to the affinity-matured antibody, comprising a step of administering to the subject a second antigen that binds to the affinity-matured antibody with greater affinity than to the germline antibody.

The invention also provides the use of a first antigen in the manufacture of a medicament for eliciting the production of an affinity-matured antibody in a subject, wherein the first antigen binds to a germline antibody with greater affinity than to the affinity-matured antibody, and wherein the subject later receives a second antigen that binds to the affinity-matured antibody with greater affinity than to the germline antibody.

The invention also provides the use of a second antigen in the manufacture of a medicament for eliciting the production of an affinity-matured antibody in a subject, wherein the medicament is administered to a subject who has previously received a first antigen that binds to a germline antibody with greater affinity than to the affinity-matured antibody.

In some embodiments the first and second antigens are administered at the same time, by simultaneous separate or sequential administration. This approach may not work in all circumstances but is suitable in situations where the presence of the second antigen does not prevent the first antigen from stimulating the desired subset of B cells. Thus the first antigen can still elicit an efficient germline antibody response that matures, due to the presence of the second antigen, into the desired affinity-matured antibody response e.g. by somatic hypermutation. For any particular situation it is straightforward to determine if the first and second antigens can function in this way or whether they need to be serially administered at separate times.

The use of a prime-boost immunization schedule is well known. For example, children typically receive a series of primary immunizations up to the age of 15 months (e.g. a DTPa vaccine) and then receive booster doses aged between 4-6 years and beyond (e.g. a Tdap vaccine). Although the priming and boosting vaccines may differ in their precise composition (e.g. the antigen ratios differ in DTPa and Tdap vaccines) antigens used in the two vaccines are typically the same. In contrast, the invention involves the use of two different antigens, one at an early stage to stimulate germline antibody production and one at a later stage to stimulate affinity-matured antibody production.

To select suitable first and second antigens the invention will typically be used in reverse i.e. it will start with an affinity-matured antibody of interest that recognises an antigen of interest. The mature antibody's genesis will be traced back to the relevant germline antibody and a first antigen will then be selected that efficiently stimulates production of this germline antibody. The antigen of interest can then be administered to stimulate maturation of this germline antibody. The invention does not require this reverse process, but this is the usual way in which things will proceed. Thus the invention also provides a method for designing an immunisation schedule for eliciting an affinity-matured antibody of interest that binds to an antigen of interest, comprising steps of: (i) identifying the affinity-matured antibody's germline origin antibody; and (ii) identifying an antigen that has higher affinity for the germline origin antibody than for the affinity-matured antibody. The antigen identified in step (ii) can be used as the first antigen and the antigen of interest can be used as the second antigen.

For example, after a catastrophic disease episode with high mortality (e.g. SARS in 2003, or an avian influenza outbreak in humans) those who survive the infection often do so because they were able to mount a protective antibody response. Serum from these patients can be analysed to find the protective antibodies and these antibodies can be produced for therapeutic purposes. Thus the authors of references 1 to 3 were able to interrogate the memory repertoire of human immune donors who had survived SARS or highly-pathogenic avian influenza and then isolate neutralizing antibodies that had been selected in the course of natural infection. Starting with such antibodies, the invention offers a way in which their progenitor germline antibodies can be traced and then, to direct an immune response towards the protective antibodies, an antigen can be selected that will preferentially lead to expansion of these germline antibodies.

For example, many broadly neutralizing antibodies (against influenza virus, HIV, ebola virus, hepatitis C virus, etc.) have been found to derive from the same VH germline antibody, namely 1-69 (including the 51p1 allele). These antibodies are generally deleted from the adult repertoire, perhaps because they are prone to self-reactivity, which may explain why viruses have not evolved to avoid their impact. Antigens can be selected which will bias the immune system towards producing antibodies derived from this germline antibody to be amplified, thereby increasing the likelihood of obtaining a neutralizing antibody.

An antibody of interest can be sequenced by standard molecular biology techniques, either by amino acid sequencing or by sequencing the antibody-encoding genes of a B cell that expresses the antibody. Alternatively, the sequence of an antibody of interest can be deduced from a high resolution structure of the antibody. This sequence information can readily be compared to germline sequences from the relevant species to determine the antibody's original germline basis, before somatic mutation took place. Antibody germline sequences can be determined from genome sequencing and are available in public databases. For instance, reference 4 introduced IMGT/GENE-DB, a comprehensive database including human and mouse antibody gene sequences.

Having established an antibody's original germline sequence, an antigen is then selected that will stimulate the expansion of B cells with this germline sequence. Various methods can be used to make this selection. For instance, a peptide library can be screened against clones of the relevant B cells. Suitable peptides from this screening can also be screened to exclude those which stimulate expansion of undesirable B cell lineages, leaving peptides that have the desired ability to stimulate expansion of B cells with the desired germline sequences. As an alternative to using B cells in these assays it is possible to use purified antibodies (e.g. recombinant antibodies) but activity of the final selected peptide(s) can be confirmed against B cells.

Another way of selecting a first antigen is to use a library of peptides (e.g. a phage display library) to pan against the germ-line antibody.

Another way of selecting a first antigen is to use a derivative of the pathogen factor that is recognised by the antibody of interest. For instance, the authors of references 2 and 3 selected patient antibodies that recognise the hemagglutinin antigen of H5 strains of avian influenza virus. This hemagglutinin antigen can be subjected to random mutagenesis and the relative abilities of the wild-type and mutagenised hemagglutinins to stimulate expansion of the germline B cells can be assayed. Molecular evolution can also be used to generate modified hemagglutinins that can preferentially stimulate the progenitor B cell. It is trivial, of course, to determine which antigen(s) in the relevant pathogen are recognised by the antibody of interest, thereby providing the starting point for mutagenesis, evolution, etc.

Another way of selecting a first antigen is to use an anti-idiotypic approach. The germline antibody produced by the progenitor B cell can be used to immunise a suitable host and anti-idiotypic antibodies can then be selected. Anti-Id antibodies from this screening can also be screened to exclude those which stimulate expansion of undesirable B cell lineages, leaving anti-Id antibodies that have the desired ability to act as antigens to stimulate B cells that produce the desired germline antibody.

Any of these methods can be used to identify a first antigen that will stimulate B cells which encode germline antibodies that can expand to provide the antibody of interest (i.e. the affinity-matured antibody) by affinity maturation. Administration of such a first antigen to a subject will result in preferential stimulation of these B cells, which will subsequently enter the affinity maturation process and undergo (in mammals) somatic hypermutation. To direct the maturation of these B cells towards those which will produce high affinity antibodies such as the original antibody of interest, a second antigen is administered to the subject. The second antigen is different from the first and will typically be the wild-type antigen that is recognised by the antibody of interest. It is not necessary to use the wild-type antigen, however, as long as the second antigen promotes the relevant affinity maturation. For example, fragments or derivatives of the wild-type antigen may be used, as may anti-Id antibodies, as described above.

A further way in which first and second antigens can be designed is to analyze the structures of complexes between the target antigen and both the germline antibody and the affinity-matured antibody, and then to use the results of this analysis in structure-based design of suitable first and second antigens.

The first and second antigens can be any suitable molecule e.g. they may comprise polypeptides, saccharides, lipids, or combinations thereof. Typically the first and second antigens will both be in the same chemical class e.g. both polypeptides, both saccharides, etc. but this will not always be necessary. In some embodiments, for instance, the first and second antigens do not have any structural or biochemical similarity but they do have the ability to function as described above e.g. the first molecule may be a small organic molecule (e.g. molecular weight<1000 Da) that suitably agonizes a useful progenitor B cell, while the second antigen is a protein.

The subject who receives the first and second antigens is a vertebrate and may be a fish, amphibian, reptile, bird or mammal. All such vertebrates have antibody responses that undergo affinity maturation, involving somatic hypermutation in mammals, gene conversion in birds, etc. The main areas of interest, however, are mammals and in particular humans. Aside from humans, however, the invention is also useful for domesticated animals (e.g. cats or dogs), other animals of commercial importance (e.g. cows, poultry, chickens, horses, pigs, fish), or wild animals (e.g. fowl, geese, ducks, chickens, foxes, bats) that can serve as reservoirs of diseases that may be transmitted to humans, to domesticated animals, or to animals of commercial importance. The methods and uses of the invention are useful for providing protective immunity in such subjects.

Antigens may be administered directly. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal or transcutaneous, intranasal, ocular, aural, pulmonary or other mucosal administration. Alternatively, the antigens may be expressed in the host by the delivery of a vector or nucleic acid that directs the production of the antigens of interest in host tissues.

A subject may receive a single dose of the first antigen or multiple doses. Similarly, a subject may receive a single dose of the second antigen or multiple doses. Typically the subject will not receive a further dose of the first antigen after a first dose of the second antigen has been administered. In a multiple dose schedule the various doses may be given by the same or different routes e.g. a parenteral prime and mucosal boost, a mucosal prime and parenteral boost, etc. Multiple doses of an antigen will typically be administered at least 1 week apart (e.g. about 2 weeks, about 3 weeks, about 4 weeks, about 6 weeks, about 8 weeks, about 10 weeks, about 12 weeks, about 16 weeks, etc.).

The invention can be used to immunize a subject against a pathogen (including bacteria, viruses, parasites, and fungi) and to protect against disease. Various such pathogens and diseases are amenable to the invention. Thus the invention can be used against pathogens including, but not limited to, C. diphtheriae, C. tetani, B. pertussis, poliovirus, hepatitis B virus, H. influenzae type B, N. meningitidis, S. pneumoniae, measles virus, mumps virus, rubella virus, rotavirus, influenza A viruses, influenza B virus, varicella zoster virus, hepatitis A virus, human papillomavirus, human immunodeficiency virus, respiratory syncytial virus, rhinovirus, parainfluenzaviruses, human metapneumovirus, human cytomegalovirus, norovirus, malaria (e.g. species of Plasmodium), Staphylococcus aureus (including MRSA strains), Clostridium difficile, Coagulase-negative Staphylococcus species (‘CoNS’, including S. haemolyticus and S. epidermidis), Candida strains (such as C. albicans), Enterococci, Klebsiella pneumoniae, Acinetobacter species, Pseudomonas aeruginosa, Streptococcus agalactiae, Streptococcus pyogenes, and extraintestinal pathogenic Escherichia coli (‘ExPEC’).

Immunogenic compositions including the first or second antigens will be pharmaceutically acceptable. Thus they will usually include components in addition to the antigens e.g. they typically include one or more pharmaceutical carrier(s) and/or excipient(s). A thorough discussion of such components is available in reference 5.

Compositions will generally be administered in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form for use.

Compositions may include one or more immunoregulatory agents such as vaccine adjuvants. Such adjuvants include, but are not limited to, insoluble metal salts (e.g. an aluminium hydroxide or aluminium phosphate adjuvant), oil-in-water emulsions (e.g. squalene-in-water emulsions with submicron oil droplets, such as MF59 or AS03), saponins, immunostimulatory oligonucleotides, ADP-ribosylating toxins and detoxified derivatives thereof, monophosphoryl lipid A (MPL) and 3-O-deacylated MPL (3dMPL), polyoxyethylene ethers and polyoxyethylene esters, phosphazenes, muramyl peptides, imidazoquinolones, substituted ureas, aminoalkyl glucosaminide phosphates, thiosemicarbazones, Tryptanthrins, Isatorabine, Loxoribine, polyoxidonium polymers, N-oxidized polyethylene-piperazines, methyl inosine 5′-monophosphate, polyhydroxlated pyrrolizidines, α-glycosylceramides gamma inulins, etc. Combinations of adjuvants may also be used.

In some embodiments of the inventions an immunoregulatory agent may be used to enhance the desired effects of the first and second antigens. Clonal expansion is stimulated by different signals from those that cause class switching and somatic hypermutation and so immunoregulatory agent(s) may be administered with the first and second antigens to provide or enhance the appropriate signals.

In some embodiments where a first antigen is followed by a second antigen the invention may use one or more intermediate antigens. The first antigen is used to stimulate the desired germline progenitor B cells and the second antigen is used to promote the desired mutated somatic cells, but in between these two steps evolution of the antibody can be directed by administering intermediate antigens e.g. in a series where the antigen in each step becomes less like the first antigen and more like the second antigen.

The practice of the present invention will employ, unless otherwise indicated, conventional methods of chemistry, biochemistry, molecular biology, structural biology, immunology and pharmacology, within the skill of the art. Such techniques are explained fully in the literature. See, e.g., references 5-12, etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

It will be understood that the invention has been described by way of example only and modifications may be made whilst remaining within the scope and spirit of the invention.

### REFERENCES

[1] Traggiai et al. (2004) Nat Med. 10(8):871-5.
[2] Simmons et al. (2007) PLoS Med. 4(5):el78.
[3] WO2007/134327.
[4] Giudicelli et al. (2005) Nucleic Acids Research 33:D256-D261.
[5] Gennaro (2000) Remington: The Science and Practice of Pharmacy. 20th edition, ISBN: 0683306472.
[6] Methods In Enzymology (S. Colowick and N. Kaplan, eds., Academic Press, Inc.)
[7] Handbook of Experimental Immunology, Vols. I-IV (D. M. Weir and C C. Blackwell, eds, 1986, Blackwell Scientific Publications)
[8] Sambrook et al. (2001) Molecular Cloning: A Laboratory Manual, 3rd edition (Cold Spring Harbor Laboratory Press).
[9] Handbook of Surface and Colloidal Chemistry (Birdi, K. S. ed., CRC Press, 1997)
[10] Ausubel et al. (eds) (2002) Short protocols in molecular biology, 5th edition (Current Protocols).
[11] Molecular Biology Techniques: An Intensive Laboratory Course, (Ream et al., eds., 1998, Academic Press)
[12]PCR (Introduction to Biotechniques Series), 2nd ed. (Newton & Graham eds., 1997, Springer Verlag)

1. A method for eliciting the production of an affinity-matured antibody in a subject, comprising steps of: (i) administering to the subject a first antigen that binds to a germline antibody with greater affinity than to the affinity-matured antibody; and then (ii) administering to the subject a second antigen that binds to the affinity-matured antibody with greater affinity than to the germline antibody, wherein the germline antibody and the affinity-matured antibody both bind to the first antigen and the second antigen, but wherein the germline antibody binds to the first antigen more tightly than it binds to the second antigen and the affinity-matured antibody binds to the second antigen more tightly than it binds to the first antigen.

2. The method of claim 1, wherein the germline antibody and the affinity-matured antibody both bind to the same epitope in the first antigen.

3. The method of claim 1, wherein the germline antibody and the affinity-matured antibody both bind to the same epitope in the second antigen.

4. The method of claim 1, wherein the subject is a mammal.

5. The method of claim 4, wherein the mammal is a human.

6. The method of claim 1, wherein the subject receives a third antigen between the first antigen and the second antigen.

7. The method of 1, wherein the germline antibody has a VH1-69 heavy chain.
